(19) United States
(12) Patent Application Publication
(10) Pub. No.: US 2006/0269569 A1
(43) Pub. Date: Nov. 30, 2006

(54) Universal antioncotic and pain relieving ointment

(76) Inventor: Liu Yuen, San Gabriel, CA (US)

Correspondence Address: Paul R. Martin, 2291 Bennington Drive, Vallejo, CA, 94591, US

(21) Appl. No.: 11/433,820

(22) Filed: May 12, 2006

Related U.S. Application Data
(60) Provisional application No. 60/681,239, filed on May 16, 2005.

(51) Int. Cl. A61K 36/254; A61K 36/537; A61K 36/328; A61K 36/48

(52) U.S. Cl. 424/195.18; 424/728; 424/748; 424/746; 424/757; 424/773; 424/769

(57) ABSTRACT

Described is a universal antioncotic and pain reducing ointment comprising an ointment formulated from the following herbs: Eucommiae Cortex, San-Chi Ginseng Radix, Radix Rehmanniac, Sparganii Rhizoma, Zedoariae Rhizoma, Angelicae Sinensis Radix, Radix Ledebouriellae, Sappan Lignum, Gardneiae Fructus, Salviae Radix, Olibanum (Gummi Olibanum), Myrrha, Lonicerae Flos, Ginseng Radix Rubra, and panacis Quinquefolii Radix

### TECHNICAL FIELD

This invention relates to an antioncotic and pain relieving/reducing ointment for application to the skin of the human body. The ointment is based on principles used in oriental medicine, utilizing herbs and other compounds. It is effective in reducing joint swelling and pain, as well as other conditions in the body and on the skin.

### BACKGROUND OF THE INVENTION

Chinese medicine is an ancient art, having been in existence for over 4,000 years. Chinese medicine is based on different principles than are used in standard Western medicine, and utilizes natural herbs in many of its medicines in order to counter the ills of the body.

Herbs are plant, mineral, and animal substances that the body assimilates through its digestive, respiratory, and cutaneous tissues. They reorganize the body constituents recognized in Chinese Medicine (Qi, Moisture, and Blood) within Organ Networks and oust the Adverse Climates that are principles of Chinese Medicine (Wind, Heat, Cold, Dryness, and Dampness). Four basic properties dispersing, consolidating, purging, and tonifying represent an herb's potential for achieving a particular outcome. Properties are deduced from observing the effects herbs evoke.

Pain in the human body arises from many causes, but a primary one is due to swelling of tissues which causes impingement on nerves, that in turn transmit pain signals to the brain. Joints in particular, are susceptible to pain as a consequence of infection, making it difficult for one having joint pain to move around effectively. Additionally, arthritis swelling and pain is a source of great discomfort as individuals get older. There are many types of arthritis that form in the joints, and all in particular cause pain when the condition becomes severe.

### BRIEF SUMMARY OF THE INVENTION

The present invention is an herbal based ointment effective in reducing swelling and pain in the human body. It is primarily useful to reduce joint pain and swelling, but is effective also in reducing swelling and pain due to injury to all tissues, specifically cuts and bruises of the skin. As used herein, the terms “reducing” and “relieving” are used interchangeably, and refer in a reduction in the level of swelling or pain incurred as a consequence of illness, infection, or injury to the human body.

### DETAILED DESCRIPTION OF THE INVENTION

The universal antioncotic and pain reducing ointment of the invention has the following formulation.
(a) from about 4 to about 20% by weight Eucommiae Cortex
(b) about 5% by weight San-Chi Ginseng Radix
(c) from about 10 to about 23% by weight Radix Rehmanrniac
(d) from about 1% to about 16% by weight Sparganii Rhizoma
(e) from about 1 to about 15% by weight Zedoariae Rhizoma
(f) from about 1 to about 16% by weight Angelicae Sinensis Radix
(g) from about 1 to about 15% by weight Radix Ledebouriellae
(h) from about 1 to about 14% by weight Sappan Lignum
(i) from about 1 to about 15% by weight Gardneiae Fructus
(j) from about 1 to about 13% by weight Salviae Radix
(k) about 2% by weight Olibanum (Gummi Olibanum)
(l) about 2% by weight Myrrha
(m) from about 1% to about 13% by weight Lonicerae Flos
(n) about 8% by weight Ginseng Radix Rubra, and
(o) about 3% by weight panacis Quinquefolii Radix

More preferably, the ointment has the following formulation:
(a) from about 6 to about 18% by weight Eucommiae Cortex
(b) about 5% by weight San-Chi Ginseng Radix
(c) from about 12 to about 21% by weight Radix Rehmanniac
(d) from about 2% to about 14% by weight Sparganii Rhizoma
(e) from about 1 to about 13% by weight Zedoariae Rhizoma
(f) from about 2 to about 14% by weight Angelicae Sinensis Radix
(g) from about 1 to about 13% by weight Radix Ledebouriellae
(h) from about 1 to about 12% by weight Sappan Lignum
(i) from about 1 to about 13% by weight Gardneiae Fructus
(j) from about 1 to about 11% by weight Salviae Radix
(k) about 2% by weight Olibanum (Gummi Olibanum)
(l) about 2% by weight Myrrha
(m) from about 1% to about 11% by weight Lonicerae Flos
(n) about 8% by weight Ginseng Radix Rubra, and
(o) about 3% by weight panacis Quinquefolii Radix

Most preferably, the ointment has the following formulation:
(a) about 12% by weight Eucommiae Cortex
(b) about 5% by weight San-Chi Ginseng Radix
(c) about 18% by weight Radix Rehmanniac
(d) about 8% by weight Sparganii Rhizoma
(e) about 7% by weight Zedoariae Rhizoma
(f) about 8% by weight Angelicae Sinensis Radix
(g) about 7% by weight Radix Ledebouriellae
(h) about 6% by weight Sappan Lignum
(i) about 7% by weight Gardneiae Fructus
(j) about 5% by weight Salviae Radix
(k) about 2% by weight Olibanum (Gummi Olibanum)
(l) about 2% by weight Myrrha
(m) about 5% by weight Lonicerae Flos
(n) about 8% by weight Ginseng Radix Rubra, and
(o) about 3% by weight panacis Quinquefolii Radix

The universal antioncotic and pain reducing ointment of this invention is prepared in the following manner. The medicinal materials are cleaned and dried. They are ground at high speed until they become a powder of approximately 120 mesh fineness. They are then mixed with sufficient rice wine at 40 degrees to make a paste. The paste is mixed with a cotton yarn to produce an adhesive patch of 0.5 mm or 0.10 mm in thickness. Each patch is then sealed in a transparent hard sealing tape.

To use, the ointment patch is removed from its package, and placed over the affected spot, then taped in place. It is taken off after 2-3 hours, and the affected spot cleaned. It can be applied as often as desired.

The efficacy of the universal antioncotic and pain relieving ointment of this invention is demonstrated in the following examples.

Case A. Mr. Chang, who lived in San Francisco, was 48 years old, and suffered from osteoproliferation and spurs on his spine at the level of the waist. He had been lying in bed, and could not get up. He suffered from tremendous pain and needed support of two people in order to get up on the bed. After applying the antioncotic and pain relieving ointment of the invention, his pain was substantially reduced, without the need of surgery.

Case B. Mrs. Zhu's co-worker, an American, fell and hurt his head. He suffered swelling and pain which affected his diet and sleep. He was in terrible pain. After Applying the antioncotic and pain relieving ointment of the invention for three days, he was cured without surgery or any oral medicine.

Case C. Mrs. Huang, age 45, twisted her ankle while she was on a cruise. When she returned from her trip, her ankle had swelled up like a pig's foot. She was in terrible pain and needed other people's support to work. Later, after applying the antioncotic and pain relieving ointment of the invention for fifteen days, she was fully recovered. Her sister also twisted her leg, and has been using other methods for treatment for three years, and she has yet to be cured.

Another preferred embodiment of the invention has the formula:
about 11% by wt. Kwangxi sancheng michiu. (Rice wine Alc.38% by vol.)
about 6% by wt. Young Ji
about 6% by wt. Eucommiae Cortex
about 5% by wt. San-Chi Ginseng Radix
about 10% by wt. Radix Rehmanniac
about 8% by wt. Sparganii Rhizoma
about 7% by wt. Zedoariae Rhizoma
about 5% by wt. Angelicae Sinensis Radix
about 7% by wt. Radix Ledebouriellae
about 6% by wt. Sappan Lignum
about 7% by wt. Gardneiae Fructus
about 5% by wt. Salviae Radix
about 2% by wt. Olibanum (Gummi Olibanum)
about 2% by wt. Myrrha
about 5% by wt. Lonicerae Flos
about 5% by wt. Ginseng Radix Rubra, and
about 3% by wt. panacis Quinuefolii Radix
   This formulation also reduces swelling and alleviates pain in the human body, when applied on the skin in the manner set forth herein.

Although this invention has been described in its preferred forms with a certain degree of particularity, it will be appreciated by those skilled in the art that the present disclosure of the preferred forms has been made only by way of example and that various changes in the percentage and specificity of ingredients in the formulation of the ointment can be made without departing from the scope and spirit of the invention.

I claim:

1. A universal antioncotic and pain relieving ointment comprising:
(a) from about 4 to about 20% by weight Eucommiae Cortex
(b) about 5% by weight San-Chi Ginseng Radix
(c) from about 10 to about 23% by weight Radix Rehmanniac
(d) from about 1% to about 16% by weight Sparganii Rhizoma
(e) from about 1 to about 15% by weight Zedoariae Rhizoma
(f) from about 1 to about 16% by weight Angelicae Sinensis Radix
(g) from about 1 to about 15% by weight Radix Ledebouriellae
(h) from about 1 to about 14% by weight Sappan Lignum
(i) from about 1 to about 15% by weight Gardneiae Fructus
(j) from about 1 to about 13% by weight Salviae Radix
(k) about 2% by weight Olibanum (Gummi Olibanum)
(l) about 2% by weight Myrrha
(m) from about 1% to about 13% by weight Lonicerae Flos
(n) about 8% by weight Ginseng Radix Rubra, and
(o) about 3% by weight panacis Quinquefolii Radix

2. A universal anitoncotic and pain relieving ointment comprising:
(a) from about 6 to about 18% by weight Eucommiae Cortex
(b) about 5% by weight San-Chi Ginseng Radix
(c) from about 12 to about 21% by weight Radix Rehmanriac
(d) from about 2% to about 14% by weight Sparganii Rhizoma
(e) from about 1 to about 13% by weight Zedoariae Rhizoma
(f) from about 2 to about 14% by weight Angelicae Sinensis Radix
(g) from about 1 to about 13% by weight Radix Ledebouriellae
(h) from about 1 to about 12% by weight Sappan Lignum
(i) from about 1 to about 13% by weight Gardneiae Fructus
(j) from about 1 to about 11% by weight Salviae Radix
(k) about 2% by weight Olibanum (Gummi Olibanum)
(l) about 2% by weight Myrrha
(m) from about 1% to about 11% by weight Lonicerae Flos
(n) about 8% by weight Ginseng Radix Rubra, and
(o) about 3% by weight panacis Quinquefolii Radix

3. A universal antioncotic and pain relieving ointment comprising:
(a) about 12% by weight Eucommiae Cortex
(b) about 5% by weight San-Chi Ginseng Radix
(c) about 18% by weight Radix Rehmanniac
(d) about 8% by weight Sparganii Rhizoma
(e) about 7% by weight Zedoariae Rhizoma
(f) about 8% by weight Angelicae Sinensis Radix
(g) about 7% by weight Radix Ledebouriellae
(h) about 6% by weight Sappan Lignum
(i) about 7% by weight Gardneiae Fructus
(j) about 5% by weight Salviae Radix
(k) about 2% by weight Olibanum (Gummi Olibanum)
(l) about 2% by weight Myrrha
(m) about 5% by weight Lonicerae Flos
(n) about 8% by weight Ginseng Radix Rubra, and
(o) about 3% by weight panacis Quinquefolii Radix

4. A method of reducing swelling and relieving pain in the human body comprising administering a therapeutically effective amount of the universal antioncotic and pain reducing ointment of claims 1,2, or 3.

5. The method of claim 4 wherein the ointment is that described in claim 3.

6. A method of reducing swelling and relieving pain in the human body comprising applying a therapeutically effective amount of the universal antioncotic and pain reducing ointment of the invention to the skin of the human body adjacent to and overlying the area of swelling and from which pain emanates.

7. The ointment of claim 1 wherein the herbal ingredients are solubilized with nice wine.

8. A universal antioncotic and pain relieving ointment comprising:
about 11% by wt. Kwangxi sancheng michiu.(Rice wine Alc.38% by vol.)
about 6% by wt. Young Ji
about 6% by wt. Eucommiae Cortex
about 5% by wt. San-Chi Ginseng Radix
about 10% by wt. Radix Rehmanniac
about 8% by wt. Sparganii Rhizoma
about 7% by wt. Zedoariae Rhizoma
about 5% by wt. Angelicae Sinensis Radix
about 7% by wt. Radix Ledebouriellae
about 6% by wt. Sappan Lignum
about 7% by wt. Gardneiae Fructus
about 5% by wt. Salviae Radix
about 2% by wt. Olibanum (Gummi Olibanum)
about 2% by wt. Myrrha
about 5% by wt. Lonicerae Flos
about 5% by wt. Ginseng Radix Rubra, and
about 3% by wt. panacis Quinuefolii Radix
